(19) United States
(12) Patent Application Publication
(10) Pub. No.: US 2008/0242942 A1
(43) Pub. Date: Oct. 2, 2008

(54) Fetal movement calculator

(76) Inventor: Patrick Tsui, San Francisco, CA (US)

Correspondence Address: PATRICK TSUI;SUITE 108, 650 CHESTNUT STREET, SAN FRANCISCO, CA, 94133-2382, US

(21) Appl. No.: 11/691,654

(22) Filed: Mar. 27, 2007

(51) Int. Cl. A61B 5/00

(52) U.S. Cl. 600/300

(57) ABSTRACT

Mother needs to calculate the fetal movement manually several times per day. No any instrument which can let mother save these jobs in the past.

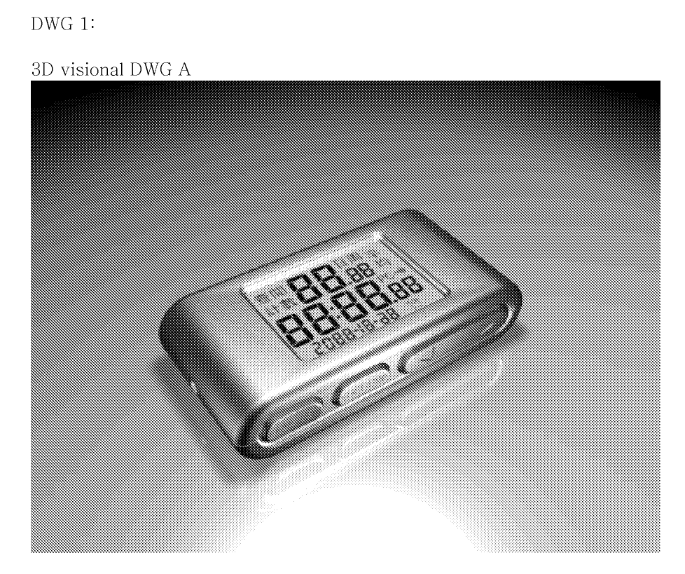

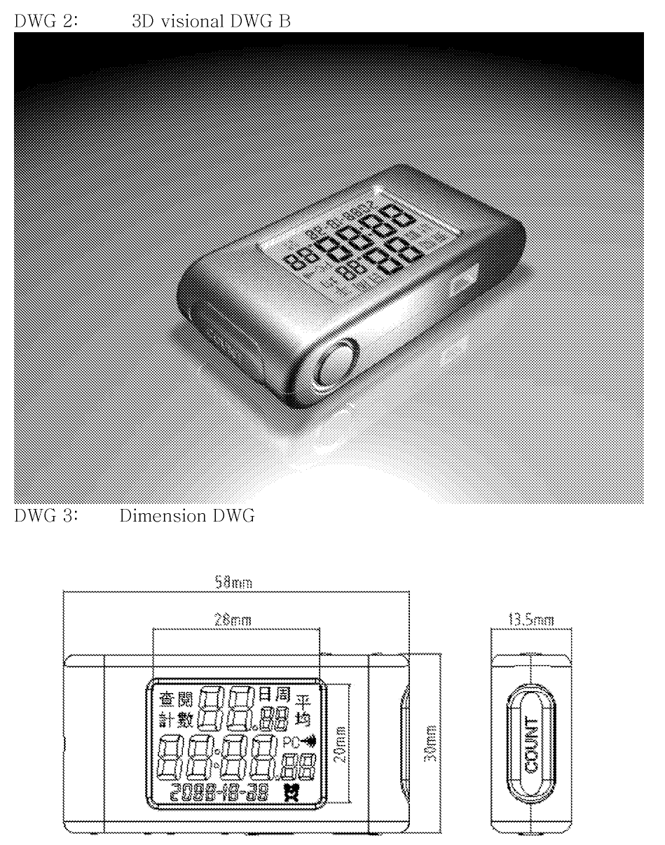

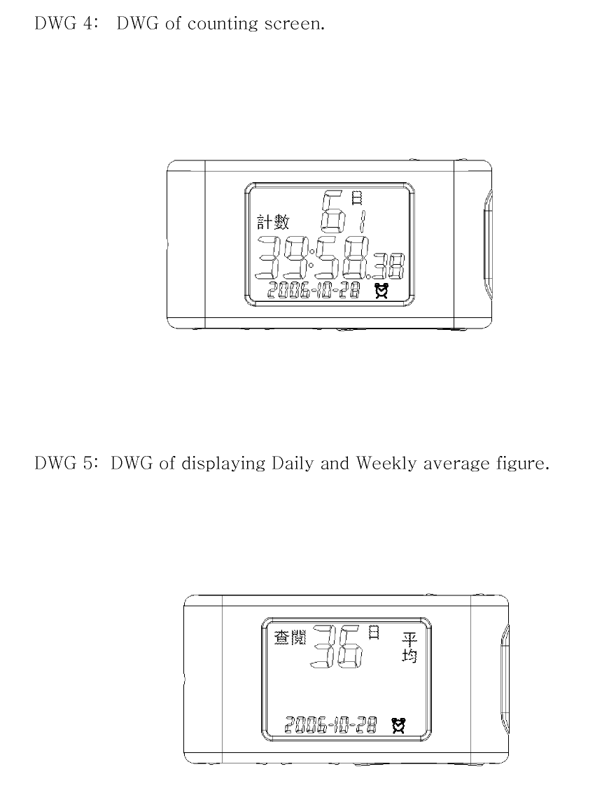

This instrument combined the function of LCD alarm clock, stop watch, and calculator to automatically calculate the daily fetal movements, and converts to the dairy and weekly average figures; and show each figure on the LCD screen.

### BACKGROUND OF THE INVENTION

The whole world obstetricians suggested the pregnant mother to constantly count the fetus's movement (also call kick movement) by themselves that is an inconvenient method and very trouble-some.

Mother needs to calculate them manually several times per day. Most mothers have no habit to do it constantly, that are not good to both mother and the fetuses.

Therefore, a special designed instrument which can let mother easily count without having them handling the complicated jobs is really necessary. That was the motivation which the inventor had for bringing this instrument to these users.

### BRIEF SUMMARY OF THE INVENTION

### Fetal Movement Calculator

This instrument combined the function of LCD alarm clock, stop watch, and calculator (also can be linked to computer for saving and printing its record).

It can automatically calculate the daily fetal movements, and converts to the dairy and weekly average figures; and show each figure on the LCD screen, so as to be printed all the data to Doctor for their judgment.

Pregnant mother needs only to push the COUNT Key as per the suggested period, (3 times per day) and leave all other jobs to be done automatically by this instrument.

This instrument is also been designed to cover every possible interrupt situation which occurs but may continue the job without breaking. Such as missing one or two times COUNT per day, or forget to COUNT some days in between etc.

### BRIEF DESCRIPTION OF THE SEVERAL VIEWS OF THE DRAWING

Fetal movement calculator LCD Screen and its buttons below:

Five buttons: START/STOP, MODE/SET, UP/DOWN, COUNTₒ NIGHT LIGHT. One USB slot for connecting to Computer.

DWG 1:

3D visional DWG A

DWG 2:

3D visional DWG B

DWG 3:

Dimension DWG

DWG 4:

DWG of counting screen.

DWG 5:

DWG of displaying Daily and Weekly average figure.

### DETAILED DESCRIPTION OF THE INVENTION

### Fetal Movement Calculator

This instrument combined the function of LCD alarm clock, stop watch, and calculator (also can be linked to computer for saving and printing its record).

It can automatically calculate the daily fetal movements, and converts to the dairy and weekly average figures; and show each figure on the LCD screen, so as to be printed all the data to Doctor for their judgment.

### (A)

Pregnant mother is suggested to hit the COUNT Key in the following 3 period daily.
First figure: Suggest collecting it in any one hour during the morning time.
Second figure: Suggest collecting it in any one hour during the afternoon time.
Third figure: Suggest collecting it in any one hour during the evening time.

### (B)

This Fetal movement calculator has been designed to automatically calculate the result as per the below formula:

(Those formula are purposely used by almost all the gynecology field.)

### Daily Average Figure:

(1)

Each daily 3 count figures been added, times 4

If forget to do it that day, jump over it without record, neither without been calculated.

(2)

If only count twice that day, add these two figures and times 6

(3)

If only count once that day, have this figure times 12.

### Weekly Average Figure:

(1)

Add all the previous 7 days “Daily average figures” and divided by 7

(2)

By moving each additional day down, it will be re-calculated the newly 7 days figures and get the new updated Weekly average figure.

Jump the missing count days, always use the previous 7 days figures for calculation

### (C)

There are three (3) different count figures per day, and a Daily average figure, a weekly average figure. Total (5) five figures on the screen.

But the first weekly average figure could only be shown before 7 counted days been collected.

If any daily average, weekly average figure is less than 30 or bigger than 40, a Bi long sound comes up for alerting.

### (D) Operation Method:

### Fetal Movement Calculator

1. Pull out the battery isolation strip, and the Fetal movement calculator will be on all the time.
2. Push and hold MODE/SET button for over 3 seconds, enter into TIME set mode. From HOUR to MINUTE to YEAR to MONTH to DATE to ALARM CLOCK.

Also use UP/DOWN key for choosing the expected figure.
3. Under ALARM CLOCK function, there is a small bell sign to be shown.
   Alarm clock sound is BI, BI, BI, BI, double sounds for one minute. To stop it, hit any key.
4. Push MODE/SET key for selecting COUNT to start Daily count.

Enter the COUNT mode.
(a) Push START/STOP key once, start COUNTING.
   Each time when mother feels the kick (movement), just hit the COUNT Key once.
(b) When reaches to one hour (have an alert sound—BI, BI, BI 3 sound for once.), the COUNT is stopping, it will not be countable if hit the button after.
(c) Under the counting period, in case there is any mistaken count, may adjust and correct it by pushing MODE/SET for 3 seconds, and use UP/DOWN to correct the right figure while the system is still operating without stopping.
(d) After this one hour counting is finished, hit and hold the START/STOP key over 3 seconds, and the counting time will go back to “0”. Also will save that data into the system. And automatically enter to the next counting for standing by. And so on.
(e) There are 3 (three) times of counting in a day (24 hours) When finishing these 3 times counting, it will automatically show a “F” signal, that means no more counting available in this date. Neither it is not workable when you hit the key for further counting.
(f) After passing 24:00 that day, it will automatically re-open the counting function from the beginning, and show “1” on the screen, and may start counting job for the next day.
5. Using TXT file for the save data.

Connect this Calculator with computer by a USB cable; the computer screen will show 3 options for selecting:

SAVE AS, PRINT, EXIT.

1. Claim the patent that covers the design which combines the function of LCD alarm clock, stop watch, and calculator to automatically calculate the daily and weekly fetal movement average figures.

2. Claim the patent which adds more accessory functions such as the remote control keys etc. based on the above (1) basic function design.

3. Claim the patent which is based the above (1) design but to calculate other physical data such as Blood pressure, pulse movement average figures etc.
